(19) United States
(12) Patent Application Publication
(10) Pub. No.: US 2017/0296401 A1
(43) Pub. Date: Oct. 19, 2017

(54) ABSORBENT ARTICLE

(71) Applicant: OJI HOLDINGS CORPORATION, Tokyo (JP)

(72) Inventors: Katsuhiko Sugiyama, Tokyo (JP); Izumi Tashiro, Tokyo (JP); Hiroyuki Hanao, Tokyo (JP); Yoshihiro Takiyama, Tokyo (JP); Toshihiro Noda, Tokyo (JP); Harue Shimizu, Tokyo (JP)

(21) Appl. No.: 15/515,957

(22) PCT Filed: Sep. 25, 2015
(86) PCT No.: PCT/JP2015/004882

§ 371 Date: Mar. 30, 2017

(30) Foreign Application Priority Data
Oct. 3, 2014 (JP) 2014-205218
Nov. 5, 2014 (JP) 2014-225418
May 15, 2015 (JP) 2015-100510

(51) Int. Cl. A61F 13/513; A61F 13/49; A61F 13/15; A61F 13/531; A61F 13/56; A61F 13/58

(52) U.S. Cl. CPC A61F 13/513; A61F 13/5655; A61F 13/49012; A61F 13/581; A61F 13/15731; A61F 13/531; A61F 2013/49092; A61F 2013/51338

(57) ABSTRACT

An absorbent article according to the present invention having a liquid-permeable top sheet, a liquid-impermeable back sheet, and an absorber disposed between the top sheet and the back sheet includes a first recessed portion configured in a top surface of the absorber facing the top sheet and extending along a first direction and a second recessed portion configured in the top surface of the absorber facing the top sheet so as to intersect the first recessed portion, the second recessed portion extending along a second direction different from the first direction. The first recessed portion and the second recessed portion each have a first area having a shallow depth from the top surface of the absorber to a bottom surface of the first area and a second area having a deeper depth than the first area.

### TECHNICAL FIELD

The present invention relates to an absorbent article having a liquid-permeable top sheet, a liquid-impermeable back sheet, and an absorber disposed between the top sheet and the back sheet.

### BACKGROUND ART

Various absorbent articles are known in which an absorber is interposed between a liquid-permeable top sheet and a back sheet, for example, diapers for infants and adults, sanitary products for women, mild or moderate incontinence pads, and excrement treatment products for pet animals. In such an absorbent article, an absorber intended to absorb and hold a liquid such as urine is mainly composed of an absorbent core that is a mixture of fluffy pulp and SAP (superabsorbent polymer), and a core wrapping material such as a tissue paper which wraps the absorbent core.

For such absorbent articles, there have been demands for lightweight and compact articles referred to as ultra-thin or ultra-ultra-thin absorbent articles in order to achieve resource saving, reduced logistic costs, improved storage efficiency at retail stores. This is becoming mainstream. The most important point to allow such an ultra-thin absorbent article to be implemented is to reduce the weight of an absorber itself that occupies most of the weight and volume of the absorbent article and to make the absorber compact. That is, a reduction in the thickness of an absorber mainly composed of a mixture of fluffy pulp and SAP has been achieved by increasing the relative content of the SAP or using a sheet-like SAP instead of the mixture.

However, in a case where an attempt is made to increase the relative content of the SAP in the absorber or to reduce the thickness of the absorber using a sheet-like SAP instead of the mixture, setting the content of the SAP higher than approximately 50 to 60% is basically difficult. This difficulty is due to a gel block phenomenon and absorption characteristics of the SAP. In particular, the reason for the difficulty is that the SAP, in spite of its high absorption holding capacity, has the property of being low in absorption speed, particularly in the startup speed of absorption. For example, a discharge speed for urine or the like varies according to individual differences, sexual differences, or age differences, but in the earliest example, 100 cc of urine is discharged within approximately 10 seconds. In contrast, even in a case where having an improved absorption speed, the SAP needs at least 30 seconds from contact of a liquid with the SAP until a state is established where the SAP fully demonstrates its absorption capacity. Due to a significant speed difference between urine discharge speed and the absorption speed at which the SAP, constituting a part of the absorber, absorbs urine, most of the urine is unabsorbed and accumulates in the absorber in a stage immediately after discharge of the urine. Free migration of the urine unabsorbed and remaining in the absorber is a major cause of leakage of the urine from the absorbent article.

Consequently, PTL 1 and the like propose a technique of suppressing leakage of urine by forming, in a surface of an absorber, a compressed recessed portion intended to promptly guide and diffuse urine from a groin area of the absorber in which urine is discharged to a front body area and a back body area of the absorber.

On the other hand, the flexibility of the absorber itself tends to be lost as the content of the SAP in the absorber is increased. The groin area of the absorber is subjected to a significant torsional force resulting from motion of a user of the absorbent article, particularly the motion of the user's legs. Thus, the loss of pliableness of the absorber increases the user's uncomfortable feeling in a case where the user puts on the absorbent article. Thus, PTL 2 and the like propose a technique in which lattice-shaped compressed grooves are configured in the surface of an absorber so that the absorber can bend easily at the portion of the compressed grooves so as to follow the torsional force applied to the absorber.

### CITATION LIST

### Patent Literature

PTL 1 Japanese Patent Laid-Open No. 2012-14535

PTL 2 Japanese Patent Laid-Open No. 2015-16218

### SUMMARY OF INVENTION

### Technical Problem

To prevent the grooves from settling even in a case where pressure is applied to the grooves due to the user's weight, the present inventor has configured compressed grooves that are unlikely to settle by compressing both the absorber and a top sheet (top sheet) arranged on a skin contact side of the absorber, from the top sheet side. Also in this case, the compressed grooves are embossed by rolling an embossing roll on the absorber with the top sheet stacked thereon. In a case where the compressed grooves are thus configured, pressing with the embossing roll allows the top sheet and the like positioned above the absorber to engage in toward the grooves. In particular, portions of an embossing pattern such as a diagonal lattice pattern which has diagonally extending components may involve a reduced distance (reduced interval) between simultaneously compressed points such as a lattice point and a point near the lattice point. The reduced distance between the simultaneously compressed points causes exertion of a strong force that pulls the top sheet at both the adjacent compressed points, leading to the likelihood that the top sheet is heavily strained.

The compressed grooves themselves are positioned lower than uncompressed areas and are thus prevented from coming into direct contact with the user's skin. However, in areas near intersection points (lattice points) in the lattice, that is, areas near corners in the lattice, the top sheet and the absorber are pulled and heavily strained and fixed. Thus, powdery SAP in the absorber may conventionally come into contact with the top sheet. Then, areas in the skin contact surface of the top sheet near the corners in the lattice are made harder than the other areas such that roughness of the high-liquid-permeability resin is transmitted to the user as a feel.

The areas near the corners in the lattice are positioned higher than the bottom of the compressed grooves and thus come into contact with the user's skin, giving the user with the feeling of hardness or coarseness. This is not preferable. Absorbent articles contact delicate skin portions such as the groin and thus need to give the user a soft feel. Hard corner portions in the lattice fail to give the user a preferable feel.

On the other hand, the top sheet is a surface contacted by the user's skin and is thus easily observed by the user while the user is putting on the absorbent article. An emboss pattern that is insufficiently excellent in terms of regularity and design produces only a low aesthetic effect and may even make the user mistake the emboss pattern for wrinkles as manufacturing defects.

An object of the present invention is to provide an absorbent article configured by compressing, along with a top sheet, a visually excellent emboss pattern having diagonally extending component so as to allow a soft feel to be given to a user even near intersection points intersected by the diagonally extending components.

### Solution to Problem

An absorbent article according to the invention having a liquid-permeable top sheet, a liquid-impermeable back sheet, and an absorber disposed between the top sheet and the back sheet comprises a first recessed portion configured in a top surface of the absorber facing the top sheet and extending along a first direction and a second recessed portion configured in the top surface of the absorber facing the top sheet so as to intersect the first recessed portion, the second recessed portion extending along a second direction different from the first direction. The first recessed portion and the second recessed portion each have a first area having a shallow depth from the top surface to a bottom surface of the first area and a second area having a deeper depth from the top surface to the bottom surface of the second area than the first area.

In the present invention, the first area having the shallow depth from the top surface to the bottom surface of the first area makes the absorber easily deformed in response to external stress such as torsion, whereas the second area having the deeper depth from the top surface to the bottom surface of the second area restrains the recessed portions from losing shapes.

The absorbent article according to the present invention may further comprise a third recessed portion in an intersection area of the first recessed portion and the second recessed portion. In this case, the third recessed portion may be discontinuous with the first recessed portion and the second recessed portion.

Furthermore, the absorber may have, in a peripheral portion thereof, a recessed portion non-forming area in which none of the first recessed portion, the second recessed portion, and the third recessed portion is configured.

The first recessed portion, the second recessed portion, and the third recessed portion may also be configured in the top sheet integrally with the absorber.

The absorber may have an undeformed area between the third recessed portion and each of the first recessed portion and the second recessed portion that are adjacent to the third recessed portion.

A plurality of the first recessed portions may be arranged at predetermined intervals along the second direction, and a plurality of the second recessed portions may be arranged at predetermined intervals along the first direction.

### Advantageous Effects of Invention

According to the absorbent article in the present invention, the first and second recessed portions configured in the top surface of the absorber have the first and second areas, respectively, having the different depths from the top surface to the bottom surface of the first and second areas. Thus, the absorbent article allows to restrict the recessed portions from losing their shapes while keeping followability to the user's body movement.

In a case where further having the third recessed portion configured in the intersection area of the first recessed portion and the recessed portion, the absorbent article allows dispersion of a liquid such as urine to be promoted. In particular, in a case where the third recessed portion is discontinuous with the first recessed portion and the second recessed portion, an area of the absorber that surrounds the third recessed portion can be kept a pleasant feel.

In a case where the absorber has, in the peripheral portion thereof, a recessed portion non-forming area in which none of the first recessed portion, the second recessed portion, and the third recessed portion is configured, this is makes it difficult for a liquid such as urine which diffuses throughout the absorber via the recessed portions to leak through the peripheral portion of the absorber.

In a case where the first recessed portion, the second recessed portion, and the third recessed portion are also configured in the top sheet integrally with the absorber, it can restrict the recessed portions from losing their shapes.

In a case where the absorber has the undeformed area between the third recessed portion and each of the first recessed portion and the second recessed portion that are adjacent to the third recessed portion, the intersection area of the first recessed portion and the second recessed portion can be kept a pleasant feel.

In a case where a plurality of the first recessed portions is arranged at the predetermined intervals along the second direction and the plurality of second recessed portions is arranged at the predetermined intervals along the first direction, the recessed portions can be configured into an aesthetically excellent lattice.

### BRIEF DESCRIPTION OF DRAWINGS

FIG. 1 is an isometric view depicting the appearance of an embodiment in which an absorbent article according to the present invention is applied to a disposable tape type diaper;

FIG. 2 is a partially broken plan view of a dispread state of the disposable tape type diaper depicted in FIG. 1 as viewed from a skin contact side;

FIG. 3 is an isometric view of the disposable tape type diaper depicted in FIG. 2 and which is in a disassembled state;

FIG. 4 is a sectional view taken along line IV-IV in FIG. 2;

FIG. 5 is a plan view of the disposable tape type diaper depicted in FIG. 1, with an absorber portion enlarged;

FIG. 6 is a plan view of the absorber depicted in FIG. 5, with a part of the absorber further extracted and enlarged;

FIG. 7 is a sectional view taken along line VII-VII in FIG. 6; and

FIG. 8 is a sectional view taken along line VIII-VIII in FIG. 6.

### DESCRIPTION OF EMBODIMENTS

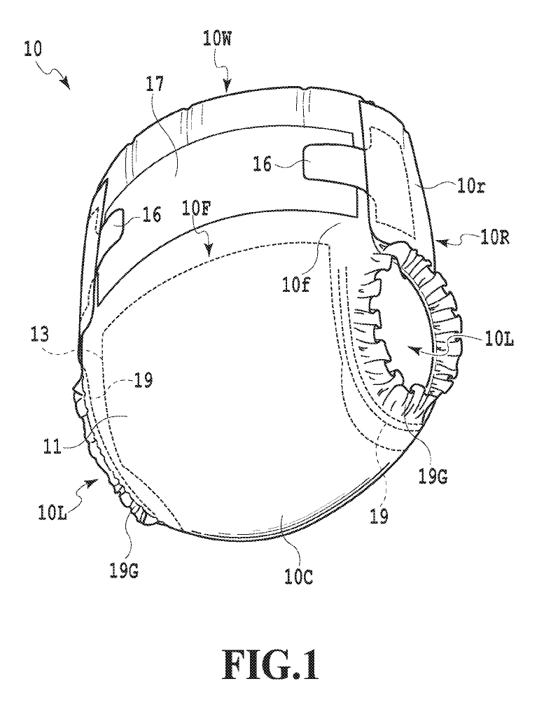

An embodiment in which an absorbent article according to the present invention is applied to a disposable tape type diaper will be described in detail with reference to FIGS. 1 to 9. However, the absorbent article according to the present invention is not limited to such a disposable tape type diaper but is applicable to various absorbent articles such as disposable pull-up type diapers and incontinence pads which have a liquid-permeable top sheet, a liquid-impermeable back sheet, and an absorber disposed between the top sheet and the back sheet.

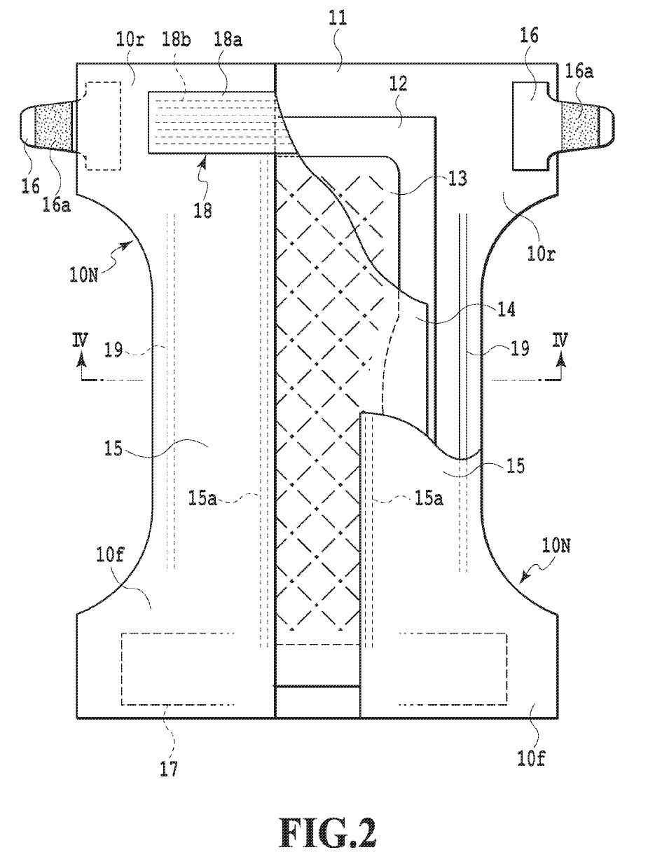

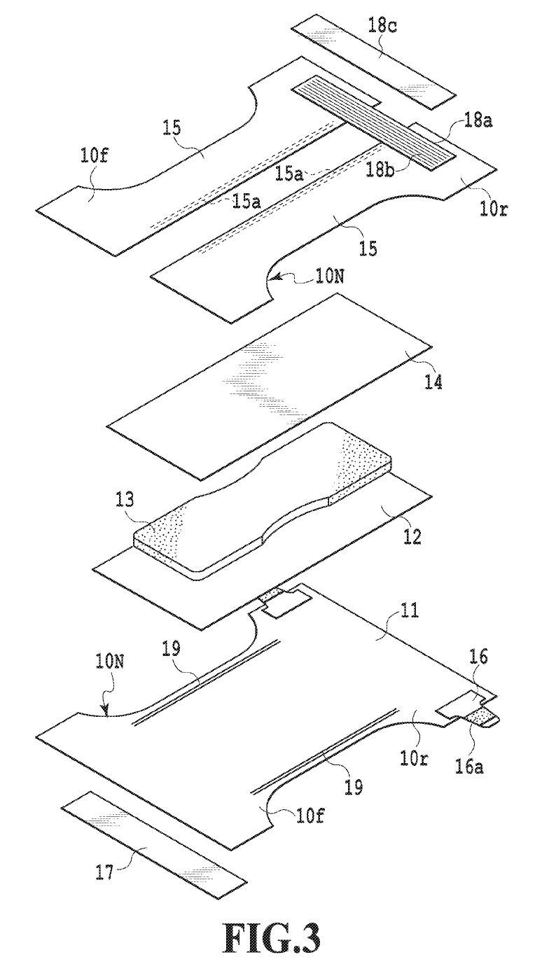

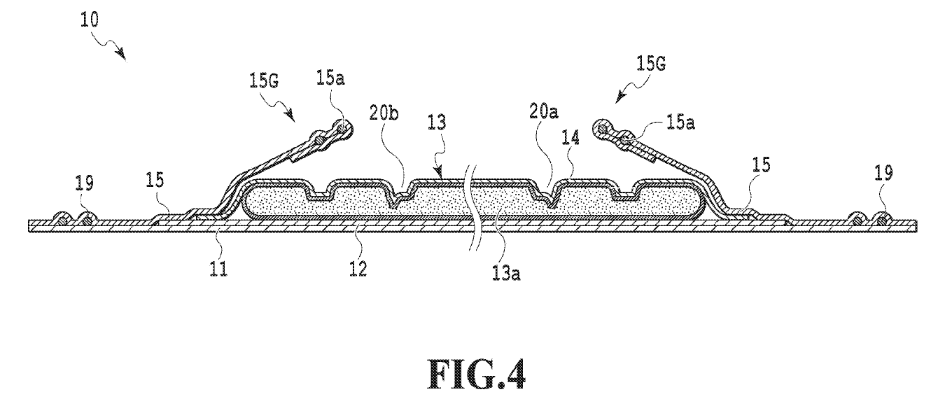

FIG. 1 is an isometric view depicting the appearance of the embodiment in which the absorbent article according to the present invention is applied to the disposable tape type diaper in usage state thereof. FIG. 2 depicts the appearance of the disposable tape type diaper dispread with an inner skin contact surface facing forward. FIG. 3 depicts the appearance of the disposable tape type diaper depicted in FIG. 2 and illustrates that the diaper is disassembled. FIG. 4 schematically depicts a sectional structure of the disposable tape type diaper as viewed along line IV-IV in FIG. 2.

That is, a tape type diaper 10 in the present embodiment has a liquid-impermeable cover sheet 11, a back sheet 12, an absorber 13, a liquid-permeable top sheet 14, and a lateral pair of side sheets 15. The back sheet 12 on which the top sheet 14 is laid has substantially the same size and shape as those of the top sheet 14 and is joined to the cover sheet 11 formed of a thin non-woven fabric in order to obtain a favorable feel. The absorber 13 is disposed between the back sheet 12 and the top sheet 14 laid on the back sheet 12. Outer peripheries of the back sheet 12 and the top sheet 14 are integrally joined together where the back sheet 12 and the top sheet 14 directly contact each other. Outer portions (in FIG. 2, lateral sides) of the liquid-impermeable side sheets 15 are laid on laterally opposite sides of the cover sheet 11 and integrally joined to laterally opposite side edges of the back sheet 12 and the top sheet 14 and to the cover sheet 11. The cover sheet 11 and the side sheets 15 have a front and a back pairs of side flap portions 10f, 10r extending leftward and rightward from the back sheet 12 and the top sheet 14, that is, extending outward from widthwise opposite edges of the back sheet 12 and the top sheet 14. In a groin area 10C between the side flap portions 10f, 10r, a pair of cutout portions 10N is configured which provides leg openings 10L for a user.

In the present embodiment, inner portions (in FIG. 2, a central side) of the top sheet 14 laid on laterally opposite sides of the top sheet 14 are not joined to the top sheet 14. To form raised gatherings 15G on the inner portions, rubber threads 15a are joined to inner edges of the side sheets 15 while being stretched.

To widthwise opposite edges of the side flap portions 10r on a back body area 10R side, base ends of a lateral pair of fastening tapes 16 are joined which are laid on the side flap portions 10f on a front body area F to connect the side flap portions together in a case where the user takes on the tape type diaper 10. In a case where the side flap portions 10r on the back body area 10R are laid on the side flap portions 10f of the front body area 10F via the fastening tapes 16, a waist opening 10W and the leg opening 10L are shaped. The fastening tapes 16 are composed of a stretchable sheet that is stretchable along a width direction of the fastening tapes 16, and have hook-and-loop fasteners 16a attached to tip portions of the fastening tapes 16. The hook-and-loop fasteners 16a can be repeatedly releasably joined to a tape fastener sheet 17 joined to the cover sheet 11 on the front body area 10F. Therefore, the fastening tapes 16 are stretched so as to exert an elastic force to join the hook-and-loop fastener 16a portions to the tape fastener sheet 17 in the front body area 10F, thus allowing an appropriate tightening force to be constantly applied to the user's waist.

Furthermore, a laterally elongate waist elastic sheet 18 disposed so as to extend across the top sheet 14 is joined to the left and right side sheets 15 on the back body area 10R side of the tape type diaper 10 while being stretched. The waist elastic sheet 18 in the present embodiment includes a base sheet 18a, a plurality of parallel rubber threads 18b joined to the bases sheet 18a while being stretched, and an upper sheet 18c joined to the base sheet 18a so as to cover the rubber threads 18b, the upper sheet 18c giving the user a favorable feel. The waist elastic sheet 18 is disposed along the waist opening 10W and functions to form waist gathering not depicted in the drawings to allow the absorbent article to appropriately fit the user's waist. Similarly, leg elastic members 19 are disposed to extend from the groin area 10C located outward of opposite side edges of the absorber 13 so as to extend along a longitudinal direction, toward the front body area 10F side and toward the back body area 10R side. The leg elastic members 19, formed of the rubber threads in the present embodiment, are fixed between the cover sheet 11 and the side sheets 15 while being stretched, and have a function to form leg gatherings 19G to allow the absorbent article to appropriately fit the user's legs.

Therefore, in a case where the user puts on the tape type diaper 10, the front body area 10F and the back body area 10R form the waist opening 10W, which surrounds the user' waist portion. Similarly, the groin area 10C, which corresponds to lower ends of the front body area 10F and the back body area 10R, forms the lateral pair of leg openings 10L, which surrounds thigh portions of the user's legs.

Now, the structure of the absorber 13 in the present embodiment will be described in detail.

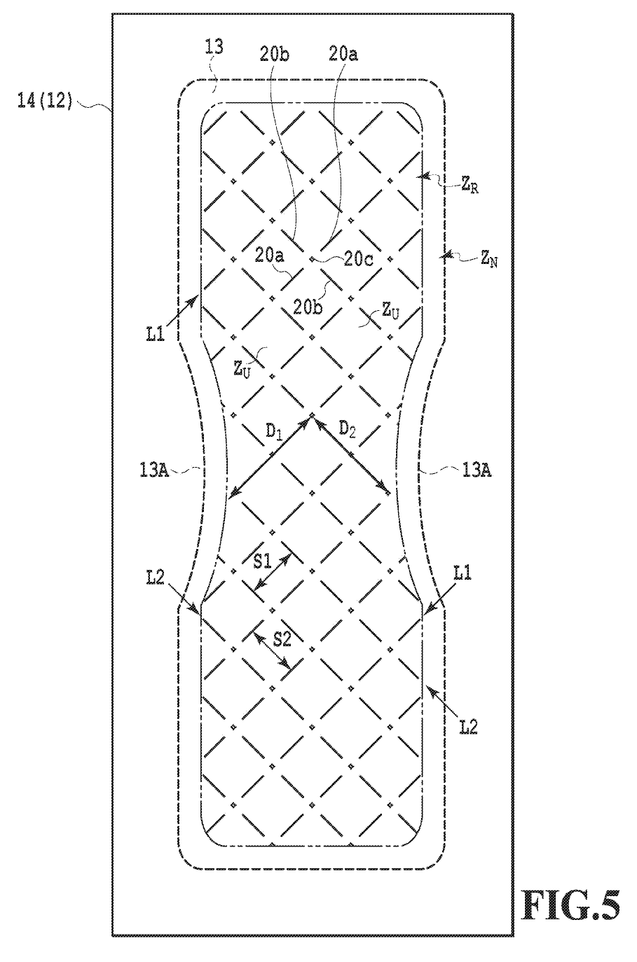

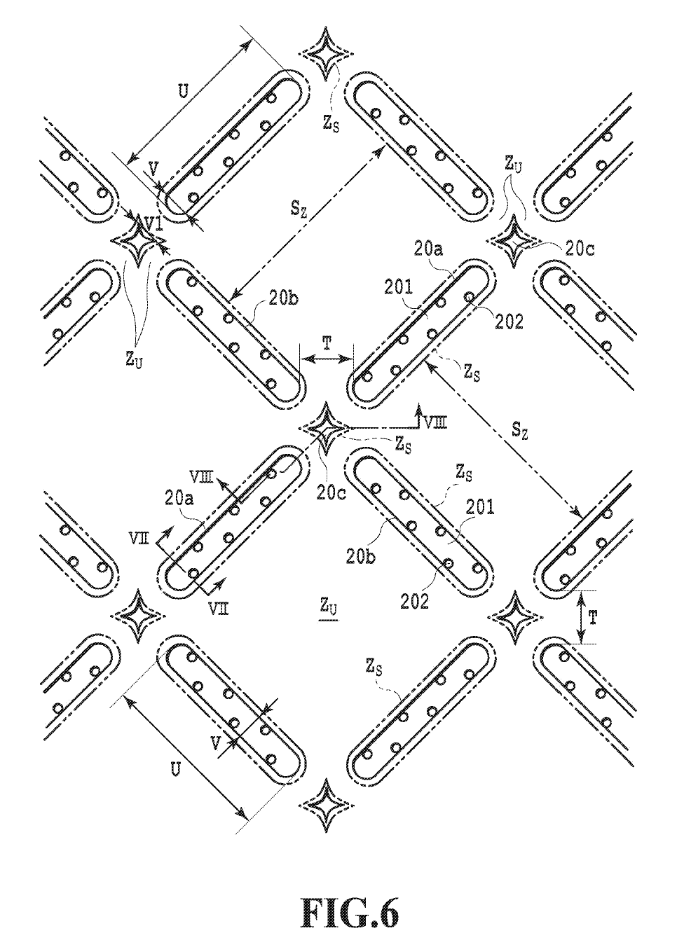

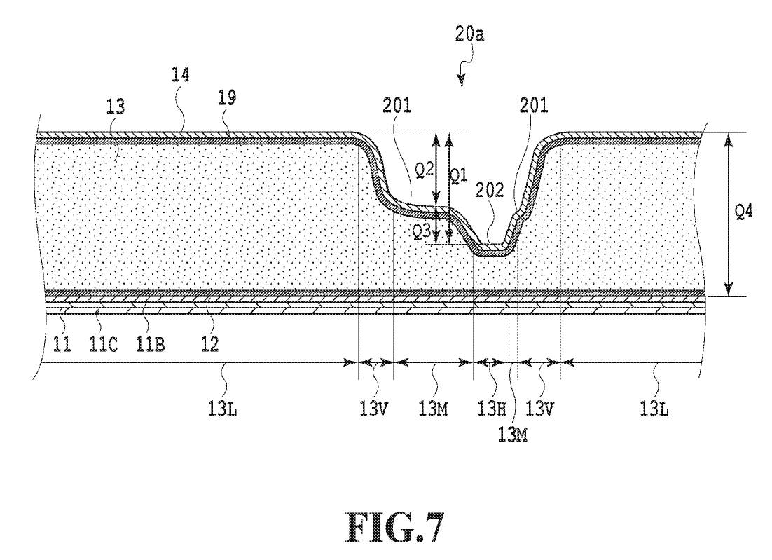

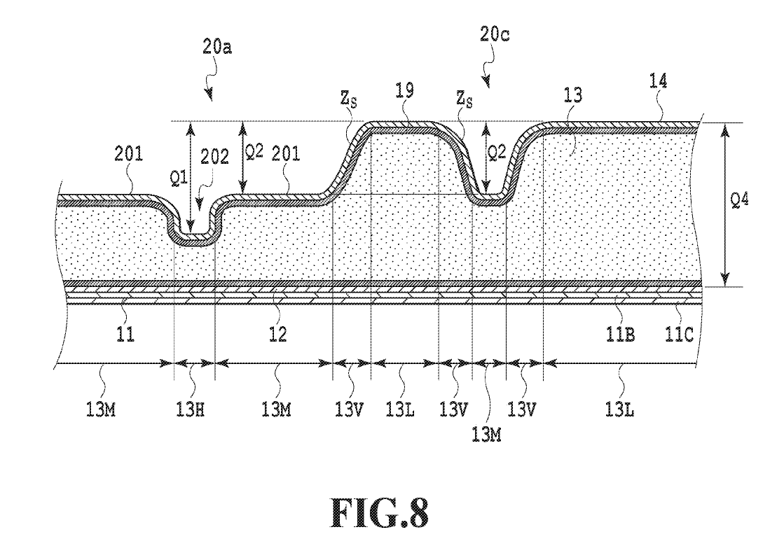

FIG. 5 depicts a planar shape of the top sheet 14, which wraps the absorber 13 between the top sheet 14 and the back sheet 12. FIG. 6 depicts a part of a top surface of the top sheet 14 extracted from FIG. 5 and enlarged. FIG. 7 and FIG. 8 are sectional views taken along line VII-VII in FIG. 5 and along line VIII-VIII in FIG. 5, respectively.

The absorber 13 in the present embodiment includes an absorbent core 13a mainly composed of pulp and SAP and a core wrap 13b such as tissue paper which wraps the absorbent core 13a. However, the absorber 13 need not necessarily include the core wrap 13b, that is, may be composed exclusively of the absorbent core 13a. The absorber 13 has a front body region and a back body region corresponding to the front body area 10F of the diaper 10 and the back body area 10R of the diaper 10, respectively, so as to extend from the front body area 10F through the groin area 10C to the back body area 10R. In a groin region of the absorber 13, a pair of arc-shaped concave portions 13A is formed in association with the lateral pair of leg openings 10L, which surrounds the thigh portions of the legs such that the groin region is smaller in width than the front body region and the back body region.

Forming the concave portions 13A in the absorber 13 is not essential. For the profile of the absorber 13, besides such an elongate shape as in the present embodiment, various shapes may be adopted such as an ellipse, a circle, or a square. Moreover, depending on the target person of using and the purpose of using, it is possible to vary the ratio of the longitudinal direction, which corresponds to an up-down direction in FIG. 5, to the width direction, which corresponds to a lateral direction in FIG. 5 which is orthogonal to the longitudinal direction.

As depicted in FIG. 5 and FIG. 6, the absorber 13 has an emboss pattern formation area regularly embossed from the top surface of the absorber 13 toward the back sheet 12 along with the top sheet 14, that is, a recessed portion forming area Z_{R}. In the recessed portion forming area Z_{R}, an emboss pattern that is generally diagonal lattice-like pattern as viewed from a distance and which includes an array of a plurality of recessed portions 20a to 20c configured by embossing. Specifically, the recessed portion forming area includes third recessed portions 20c positioned at respective intersection points in a lattice, first recessed portions 20a each positioned between the third recessed portions 20c and extending along a first direction D1, and second recessed portions 20b each positioned between the third recessed portions 20c and extending along a second direction D2 different from the first direction D1. In the present embodiment, the first direction D1 is inclined at 45 degrees to the width direction of the absorber 13, the second direction D2 is inclined at 45 degrees to the width direction of the absorber 13 in a direction opposite to the first direction D1, and thus, the emboss pattern in FIG. 5 is laterally symmetric. However, the present invention is not limited to this. It is effective to set the first and second directions D1, D2 at 10 degrees or more and less than 80 degrees to the width direction of the absorber 13. It is preferable to set the first and second directions D1, D2 at 30 degrees or more and 45 degrees or less to the width direction of the absorber 13. The third recessed portions 20c are discontinuous with the first recessed portions 20a and the second recessed portions 20b. A periphery of each of the third recessed portions 20c is surrounded a recessed portion non-forming area that is not embossed, that is, an undeformed area Z.

The diagonal lattice-like emboss pattern in the present embodiment is formed using a row of first recessed portions L1 each including the first recessed portions 20a and the third recessed portions and a row of second recessed portions L2 each including the second recessed portions 20b and the third recessed portions 20c. The row of first recessed portions L1 each include the first recessed portions 20a and the third recessed portions 20c alternately arranged along the first direction D1, and are arranged at constant intervals S2 along a direction D2. Furthermore, the row of second recessed portions L2 each include the second recessed portions 20b and the third recessed portions 20c alternately arranged along the second direction D2, and are arranged at constant intervals S1 along a direction D1. In the present embodiment, the interval S1 between the row of second recessed portions L2 along the first direction D1 and the interval S2 between the row of first recessed portions L1 along the second direction D2 are each set to 27.0 mm. However, these intervals are each preferably 110.0 mm or more and 55.0 mm or less. However, the intervals S1, S2 need not necessarily be set to the same value throughout the absorber 13 and may have values varying according to each region of the absorber 13. Furthermore, the third recessed portions 20c may be omitted from the intersection areas in the lattice, which may remain the undeformed areas Z_{U}.

The first recessed portions 20a, the second recessed portions 20b, and the third recessed portions 20c are configured by integrally compressing the top sheet 14, the core wrap 19, and the absorber 13. Furthermore, as depicted in FIG. 5, no emboss pattern is configured in a peripheral portion of the absorber 13. Therefore, the recessed portion forming area Z_{R} is surrounded by the recessed portion non-forming area Z_{N}, in which no emboss pattern is configured. The presence of the recessed portion non-forming area Z_{N} allows a liquid such as urine to be restrained from leaking from the peripheral portion of the absorber 13 through the first recessed portions 20a, the second recessed portions 20b, and the third recessed portions 20c. FIG. 5 depicts a boundary between the recessed portion forming area Z_{R} and the recessed portion non-forming area Z_{N} using a long dashed double-short dashed line.

As seen in FIG. 5 and FIG. 6, the unit cells (rhombuses) forming the lattice pattern is configured as follows. Major portions of four sides of the cell are each defined by the first recessed portion 20a or the second recessed portion 20b. Four corners of the cell are defined by the third recessed portions 20c. A length U of each of the first recessed portions 20a and the second recessed portions 20b is shorter than each of the above-described intervals S1, S2. As depicted in FIG. 5, each of the first recessed portions 20a does not overlap any of the second recessed portions 20b.

In FIG. 6, an area surrounding the periphery of each of the recessed portions 20a to 20c and depicted by a long dashed double-short dashed line is a recessed portion semi-forming area Z_{S}. That is, forming the recessed portions 20a to 20c by embossing causes the top sheet 14 and the absorber 13 to be strongly compressed such that, in conjunction with the compression, portions of the top sheet 14 and the absorber around the recessed portions 20a to 20c are pushed and shrunk. Thus, with respect to the first recessed portions 20a, the second recessed portions 21b, and the third recessed portions 20c strongly compressed with an embossing die, areas around the first recessed portions 20a, the second recessed portions 21b, and the third recessed portions 20c that are weakly compressed are hereinafter referred to as recessed portion semi-forming areas Z_{S}. Therefore, portions of the absorber 13 that are substantially deformed by embossing with respect to a state prior to embossing include not only the first recessed portions 20a, the second recessed portions 20b, and the third recessed portions 20c but also the recessed portion semi-forming areas Z_{S}. The portions of the absorber 13 other than the recessed portions 20a to 20c and the recessed portion semi-forming areas Z_{S} are no compressed by embossing and are thus undeformed areas Z_{U}. That is, the areas each between the third recessed portion 20c and each of the first recessed portion 20a and the second recessed portion 20b and the areas each enclosed by the lattice are the undeformed areas Z_{U} and are portions that accomplish the original function of the absorber 13 to hold a liquid such as urine.

In the recessed portion non-forming area Z_{N} and the undeformed areas Z_{U} in the present embodiment, the thickness (height) of the absorber 13 before embossing is substantially the same as the thickness (height) of the absorber 13 after embossing as depicted in FIG. 5 and FIG. 6. On the other hand, in the first recessed portions 20a, the second recessed portions 20b, and the third recessed portions 20c in the recessed portion forming area Z_{R}, the absorber 13 is compressed by embossing and is thinner than in the undeformed areas Z. Each of the recessed portion semi-forming areas Z_{S} connects the recessed portion forming area Z_{R} to the corresponding one of the undeformed areas Z_{U}. In the recessed portion semi-forming area, the thickness of the absorber 13 decreases gradually.

Now, the shape of each of the recessed portions 20a to 20c will be specifically described in detail.

As depicted in FIGS. 6 to 8, the first recessed portions 20a and the second recessed portions 20b each include a shallow recessed portion 201 forming the contour of the recessed portion and circular deep recessed portions 202 discontinuously disposed in the shallow recessed portion 201 and recessed more deeply than the shallow recessed portion 201. As depicted in FIG. 7, the elongate shallow recessed portion 201 is shaped like an elongate oval having a length U shorter than a distance S_{Z} between two recessed portion semi-forming areas Z_{S} opposite to each other in the first direction D1 or the second direction D2 and having a substantially constant width V. The deep recessed portions 202 each having a diameter smaller than the width V of the shallow recessed portion 201 are disposed at predetermined intervals along a longitudinal direction of the shallow recessed portion 201 so as to be alternately inscribed in opposite side wall parts of the shallow recessed portion 201. The deep recessed portions 202 are deeply recessed so as to be closer to the back sheet 12 side than the shallow recessed portion 201.

As depicted in FIG. 8, each deep recessed portion 202 is positioned so as to extend from the top surface of the top sheet 14 in the undeformed area Z_{U} to the farthest (deepest) point on the back sheet 12 side, and the shallow recessed portion 201 is positioned between the top surface of the top sheet 14 and the deep recessed portion 202 in the undeformed area Z_{U}. The depth from a bottom surface of the shallow recessed portion 201 to a bottom surface of the deep recessed portion 202, that is, a difference Q3 between a depth Q1 from the top surface of the top sheet 14 to the bottom surface of the deep recessed portion 202 in the undeformed area Z_{U} (hereinafter simply referred to as the depth of the shallow recessed portion 201) and a depth Q2 from the top surface of the top sheet 14 to the bottom surface of the deep recessed portion 202 in the undeformed area Z_{U} (hereinafter simply referred to as the depth of the deep recessed portion 202), is approximately 3.5 to 15% of the depth Q1 of the shallow recessed portion 201. Furthermore, in a case where a distance from the top sheet 14 to the back sheet 12 in the undeformed area Z_{U}, that is, the thickness of the absorber 13, is denoted as Q4, the thickness Q1 of the shallow recessed portion 201 is approximately 42.5% to 97.5% of the thickness Q4. Similarly, the depth Q2 of the shallow recessed portion 201 is approximately 37.5% to 95.0% of the thickness Q4. As described above, the first recessed portions 20a and the second recessed portions 20b in the present embodiment are configured by compressing the absorber 13 relatively deeply. Then, the absorber 13 has a two-level depth.

On the other hand, a depth from the top surface of the top sheet 14 in the undeformed area Z_{U} to a bottom surface of the third recessed portion 20c in the intersection area in the lattice (hereinafter referred to as the depth of the third recessed portion 20c) is set the same as the depth Q2 of the shallow recessed portion 201.

More specifically, the thickness of the undeformed area Z_{U} in the absorber 13 is 8.0 mm but is preferably within the range from 5.0 mm to 20.0 mm. As depicted in FIG. 8, the depth Q1 of the shallow recessed portion 201 is 7.8 mm but is preferably within the range from 3.0 mm to 8.0 mm. Preferably, the depth of each deep recessed portion 202 is 7.5 mm, and the difference Q3 between the depth Q1 of the shallow recessed portion 201 and the depth Q2 of the deep recessed portion 202 is within the range from 0.1 mm to 0.5 mm.

In the embossing that forms the first recessed portions 20a, the second recessed portions 20b, and the third recessed portions 20c, an adhesive is interposed between the top sheet 14 and the absorber 13, and both the top sheet 14 and the absorber 13 are compressed from the top surface of the top sheet 14. The first recessed portions 20a, the second recessed portions 20b, and the third recessed portions 20c are configured by compressing both the top sheet 14 and the absorber 13 from the top surface of the top sheet 14 using a predetermined die configured in an embossing roll.

In the first recessed portions 20a and the second recessed portions 20b, each of the deep recessed portions 202 is a small circle and is small in area. Therefore, pressing with the embossing roll concentrates pressure on the deep recessed portions 202 to strongly compress the absorber 13 and the top sheet 14. During the compression, pulp fibers in the absorber 13 firmly tangle with fibers in the top sheet 14 to integrally join the absorber 13 and the top sheet 14 together. The same pressing force is also applied to the shallow recessed portion 201 during the compression. However, the large area of the shallow recessed portion 201 makes the pressure less concentrated on the shallow recessed portion 201 than on the deep recessed portion 202. Thus, the junction between the absorber 13 and the top sheet 14 is slightly weaker at the shallow recessed portions 201 than at the deep recessed portions 202 but is sufficient to form the shape of the shallow recessed portion 201. As described above, the absorber 13 is strongly compressed at the deep recessed portions 202, and the absorber 13 and the top sheet 14 are firmly coupled together. Thus, the shapes of the first recessed portions 20a and the second recessed portions 20b are maintained. For example, in a case where the user of the diaper 10 is seated to apply a pressure resulting from the user's weight to the top surface of the absorber 13, the first recessed portions 20a and the second recessed portions 20b are prevented from settling, with the shapes of the first recessed portions 20a and the second recessed portions 20b maintained. Even in a case where the diaper 10 is strongly pulled by various motions of the legs, the firm junction between the top sheet 14 and the absorber 13 allows the shapes of the first recessed portions 20a and the second recessed portions 20b to be maintained.

Here, instead of the two-step structure with the shallow recessed portions 201 and the deep recessed portions 202, a structure may be configured by applying a strong compressive force to the entire first recessed portions 20a and the entire second recessed portions 20b. That is, in the embossing, the compression may be performed using a die having only generally elliptic protrusions on a surface thereof instead of using a die having generally elliptic protrusions corresponding to the shallow recessed portions 201 and round protrusions corresponding to the deep recessed portions 202 and protruding into the generally elliptic protrusions. However, such a die has no pressure concentration portions, and junctions with the fibers in the top sheet 14 firmly tangled with the fibers in the absorber 13 fail to be created unless a strong pressure is exerted all over the top sheet 14 and the absorber 13. This results in the need for a very strong pressing force, possibly causing the top sheet 14 to be torn. Compression with a generally weak pressing force has difficulty forming the first recessed portions 20a and the second recessed portions 20b that withstand the user's weight and various motions. In the present embodiment, an embossing die is provided with protrusions corresponding to the deep recessed portions 202, and partial strong compression is performed to create areas with the top sheet 14 and the absorber 13 firmly joined together and to prevent possible defects during manufacturing such as tear of the top sheet 14.

Moreover, the third recessed portions 20c disposed in the intersection areas in the lattice are configured by pressing and compressing the absorber 13 to the same degree as that for the shallow recessed portion 201 using an embossing roll as described above. The intensity of the pressing is such that the integral junction of the top sheet 14 and the absorber 13 is achieved without tear of the top sheet 14.

As depicted in FIG. 7 and FIG. 8, in the absorber 13, the bottom portion of each deep recessed portion 202 is a high-density section 13H in which the absorber 13 has the highest density, and each undeformed area Z_{U} is a low-density section 13L in which the absorber 13 has the lowest density. Furthermore, the bottom surface portion of the shallow recessed portion 201 and the bottom surface portion of the third recessed portion 20c are each as medium-density section 13M having an intermediate density between the high-density section 13H and the low-density section 13L. Moreover, each recessed portion semi-forming area Z_{S} is a density varying section 13V having a density decreasing toward the corresponding undeformed area Z_{U}.

As described above, the absorber 13 is mainly composed of SAP and pulp, and the density mainly involves the density of pulp fibers. Therefore, in the high-density section 13H, pulp is compressed to reduce the gaps between pulp pieces, whereas the gaps between pulp pieces in the low-density section 13L are larger than the gaps between pulp pieces in the high-density section 13H.

In addition, since the recessed portions 20a to 20c are configured by compressing and joining the top sheet 14, which is a synthetic resin, and the absorber 13, which is mainly composed of pulp, not much liquid such as urine tends to be absorbed to the high-density section 13H through the bottom surfaces of the recessed portions 20a to 20c. Therefore, the liquid flowing into the recessed portions 20a to 29c is absorbed to the density varying sections 13V through side walls of the recessed portions 20a to 20c and further flows to the low-density section 13L of the undeformed area Z_{U}.

In the present embodiment, in a case where the rows of first and second recessed portions L1, L2 are configured, the recessed portions 20a to 20c are discontinuously configured instead being continuously configured. In particular, the third recessed portions 20c are provided in the intersection areas in the diagonal lattice, and the first recessed portions 20a and the second recessed portions 20b surrounding each third recessed portion 20c are positioned away from the third recessed portion 20c. The reason why such a gap surrounding the third recessed portion 20c, that is, the undeformed area Z_{U}, is configured will be explained below.

As described above, the rows of first and second recessed portions L1, L2 in the diagonal lattice are configured by pressing, against the top sheet 14 and the absorber 13, an embossing roll with an emboss pattern with a complementary shape for the recessed portions 20a to 20c while rotating the embossing roll. As depicted in FIG. 5, in a case where, with the embossing roll rotated, the absorber 13 is fed in a longitudinal direction of the embossing roll, the first recessed portions 20a and the second recessed portions 20b arranged in the width direction of the absorber 13 orthogonal to a conveying direction of the absorber 13 (in FIG. 5, upward) are simultaneously pressed against the embossing roll. In the present embodiment, for the circumferential length of the embossing roll, the diameter of the embossing roll is set so as to correspond to the length of the back sheet 12 and the top sheet 14 to be cut. However, the present invention is not limited to this.

In the present embodiment, the rotating axis of the embossing roll is set orthogonal to the conveying direction of the continuous body of the back sheet 12 and the top sheet 14 with the absorber 13 disposed at a given distance from the back sheet 12 and the top sheet 14. As described above, the top sheet 14 and the absorber 13 are compressed together using the embossing roll, and then, the cover sheet 11, the side sheets 15, and the like are laid on and joined to the top sheet 14 and the absorber 13. The resultant structure is cut into the diaper 10 with predetermined dimensions.

The first recessed portions 20a are inclined 45 degrees right to the conveying direction of the absorber 13, and the second recessed portions 20b are inclined 45 degrees left to the conveying direction of the absorber 13. Thus, as the absorber 13 is fed in with respect to the embossing roll, the distance between the first recessed portion 20a and the second recessed portion 20b adjacent to each other in the width direction of the absorber 13 decreases toward the corresponding intersection point in the lattice.

In a case where pressing is performed using the embossing roll used to form the recessed portions 20a to 20c, the top sheet 14 is drawn into the recessed portions 20a to 20c. In particular, since the first recessed portion 20a and the second recessed portion 20b are disposed adjacent to each other in the width direction of the absorber 13, the top sheet 14 is laterally drawn in a case where the first recessed portion 20a and the second recessed portion 20b are configured. In other words, a widthwise (in FIG. 6, the lateral direction) drawing force is applied to the top sheet 14 between the first recessed portion 20a and the second recessed portion 20b that are adjacent to each other such that the rightward force and the leftward force are simultaneously exerted on the top sheet 14. A stronger tensile force is applied to the top sheet 14 between the first recessed portion 20a and the second recessed portion 20b as the distance between the top sheet 14 between the first recessed portion 20a and the second recessed portion 20b that are adjacent to each other decreases, that is, the distance to the corresponding intersection point in the lattice decreases.

For example, in a case where a lattice pattern with the same size is configured, in a case where the third recessed portions 20c are omitted and the first recessed portions 20a and the second recessed portions 20b are extended to positions closest to the corresponding intersection points in the lattice, the following problem may occur. That is, an area near each intersection point is hard because the absorber 13 itself has a high density in this area, and the drawing force resulting from the embossing of the neighboring left and right recessed portions 20a, 20b causes the top sheet 14 to be strongly pressed against the absorber 13. Thus, near the intersection points in the lattice, the SAP contained in the absorber 13 come into hard contact with the top sheet 14, resulting in the feeling of roughness. This stimulates the user's skin and is not preferable.

Therefore, in the present embodiment, the distance between the first recessed portion 20a and the second recessed portion 20b that are adjacent to each other in the width direction of the absorber 13 is kept at a certain value or larger to prevent the top sheet 14 from being further strongly pulled. That is, to allow simple elastic deformation of the top sheet 14 instead of plastic deformation thereof during stretching, the compression of the absorber 13 and the junction between the absorber 13 and the top sheet 14 are flexibly performed. This restrains the feeling of roughness attributed to the SAP and hardening of the intersection areas in the lattice.

The shortest distance between the first recessed portion 20a and the second recessed portion 20b along the width direction of the absorber 13 which distance satisfies the above-described state is denoted as T for convenience. In a case where the shortest distance between the first recessed portion 20a and the second recessed portion 20b along the width direction of the absorber 13 is set to T or larger, the top sheet 14 is fixed to the absorber 13 with no strong tensile force applied to the top sheet 14. Thus, the feeling of hardness can be avoided. The above-described problem with an excessive tensile force applied to the top sheet 14 does not occur in the longitudinal direction of absorber 13. However, the problem with hardening of the absorber 13 fails to be solved. Therefore, it is effective to also set the shortest distance between the first recessed portion 20a and the second recessed portion 20b along the longitudinal direction of the absorber 13 T or larger.

In other words, in a case where the emboss pattern is set, the length U of the recessed portions 20a, 20b is set such that, in a direction parallel to each of the diagonals of the lattice cells partitioned by the rows of first and second recessed portions L1, L2, the distance between the first and second recessed portions 20a, 20b that are adjacent to each other is equal to or larger than T.

By way of example, in a case where the absorber 13 is 8 mm in thickness and the third recessed portion is 6 mm in depth Q2, the above-described T is approximately 7 mm. This value varies in accordance with a relation between the amount of SAP and the amount of pulp fibers in the absorber 13 and the thickness of the top sheet 14. An important point of the present invention is that, in a case where an emboss pattern such as a diagonal lattice-shaped pattern which has diagonal components is used, the emboss pattern is not configured in portions in which the distance between the adjacent recessed portions is short.

On the other hand, the intersection points in the lattice are not where the neighboring recessed portions are pressed such that the rightward force and the leftward force are simultaneously exerted on the recessed portions. Thus, at the intersection points, the top sheet 14 is prevented from being strongly pulled. In a case where, with tension acting on the top sheet 14 taken into account, excessively large undeformed areas Z_{U} are provided, the rows of first and second recessed portions L1, L2 are prevented from being identified as rows and from being recognized as a lattice pattern. Furthermore, in a case where the distance between the third recessed portion 20a and each of the second recessed portion 20b and the third recessed portion 20c is excessively long, in other words, in a case where the undeformed areas Z_{U} are excessively large, the effect in which the absorber 13 follows the body motion and is easy to fold obliquely is difficult to produce. Therefore, the third recessed portions 20c are configured at the intersection areas in the lattice where the tension acting on the top sheet 14 is unaffected, allowing the rows of first and second recessed portions L1, L2 to be more appropriately recognized as a lattice to further enhance the visual effect of the lattice. Furthermore, the absorber 13 can be allowed to follow the user's body motion and made easy to fold without the need to harden the intersection areas in the lattice.

As described above, in the present embodiment, the distance between the adjacent recessed portions 21 is prevented from being smaller than T. In other words, in a case where the emboss pattern is configured such that the recessed portions 21 are arranged at the distance T from each other, that is, embossing is omitted from a vicinity 26 of each intersection point in the lattice, the skin contact surface is prevented from giving the feeling of hardness. The intersection areas in the lattice are fluffy like the undeformed areas Z_{U} of the absorber 13, and thus, even with a diagonal lattice-shaped emboss pattern, the intersection areas in the lattice are prevented from giving the feeling of hardness and sharpness and can give the user the feeling of fluffiness.

Furthermore, the third recessed portions 20c are provided at the intersection points of the row of first recessed portions L1 and the row of corresponding second recessed portions L2 to visually clarify the intersection points (vertexes of the lattice cells) in the lattice, improving the diaper in terms of aesthetics and design. Additionally, the diagonally extending rows of recessed portions L1, L2 can be prevented from being mistaken for wrinkles as manufacturing defects, irregularly arranged kinks, and the like. In addition, the diaper is easily deformed in a diagonal direction of the absorber 13 by following the body motion. However, the third recessed portions 20c can be omitted, and in this case, the first recessed portions 20a and the second recessed portions 20b are preferably increased in length so as to lie closer to the corresponding intersection areas in the lattice.

The length of each of the first recessed portions 20a and the second recessed portions 20b in the present embodiment is 19.0 mm, but is preferably 55% to 85% of each of the interval S1 between the row of second recessed portions L2 and the interval S2 between the row of first recessed portions L1. Furthermore, the width V of each of the first recessed portions 20a and the second recessed portions 20b is 3.0 mm, but is preferably 2.0 mm or more and 4.0 mm or less. Additionally, the width V1 of each of the third recessed portions 20c along the first direction D1 or the second direction D2 is the same as or smaller than the width V of each of the first recessed portions 20a and the second recessed portions 20b. In the present embodiment, the width V1 is set to 2.0 mm, but is preferably 1.0 mm or more and 4.0 mm or less. On the other hand, the clearance between the third recessed portion 20c and each of the first and second recessed portions 20a and 20b, that is, the length of each of the undeformed areas Z_{U}, is 4.4 mm, but is preferably 2.0 mm or more and 6.0 mm or less.

In a case where the recessed portions 20a to 20c are configured to have such depths and intervals, the grooves can be maintained even in a case where the weight is applied, and a soft feel can be maintained on the skin contact surface of the groin region. Therefore, the diagonal lattice-shaped emboss pattern allows prevention of deformation of the diaper such as kink with respect to various motions of the legs and gives a soft feel to enable stimuli to the skin to be minimized.

In the present embodiment, the external shape of each of the first recessed portions 20a and the second recessed portions 20b is defined by the elongate, generally elliptic shallow recessed portion 201. However, the present invention is not limited to this. A diagonal lattice-shaped compression row may be configured by discontinuously arranging a plurality of dots or rectangular parallelepipeds. That is, in the present embodiment, it is important to form the vicinities 26 of the intersection points such that each of the vicinities 26 has a predetermined size. The shapes of the recessed portions defined by the first recessed portions 20a and the second recessed portions 20b forming the sides of the lattice cells are not limited to the shapes in the present embodiment. Similar effects are produced even by arranging the above-described plurality of dots or the like. Even in this case, the recessed portions are arranged such that the distance between the adjacent recessed portions in the direction parallel to the diagonals of the lattice cells in the lattice is equal to or larger than T.

Furthermore, in the present embodiment, instead of one first recessed portion 20a and one second recessed portion 20b, two or more first recessed portions 20a and two or more second recessed portions 20b each of which is reduced in the length in the longitudinal direction may be used such that one side of the lattice cell is configured of the two or more recessed portions. Furthermore, the deep recessed portions 202 are arranged in the shallow recessed portion 201 such that the deep recessed portions 202 and pieces of the shallow recessed portion 201 are alternately distributed on the outer sides in the width direction. Additionally, the deep recessed portions 202 are not limited to the shape illustrated in the present embodiment but may have various shapes such as a quadrangle, an ellipse, and a triangle.

Furthermore, in the present embodiment, the third recessed portions are shaped like quadrangular bobbins. However, the present invention is not limited to this, and the third recessed portions may be barrel-like quadrangles or circles. That is, any shape may be used so long as the shape allows the third recessed portions to be recognized as intersection points in a lattice in a lattice pattern.

As described above, the present invention need to be interpreted based only on the matters recited in the claims of the invention. For the above-described embodiment, any changes or modifications included in the concepts of the present invention are possible besides the recited matters. In other words, all the matters in the above-described embodiment are not intended to limit the present invention, and the matters, including configurations not directly related to the present invention, may be optionally varied in accordance with the application or purpose of the embodiment.

### REFERENCE SIGNS LIST

10 Tape type diaper (diaper)
10C Groin area
10F Front body area
10L Leg opening
10N Cutout portion
10R Back body area
10W Waist opening
10F, 10r Side flap portion
11 Cover sheet
12 Back sheet
13 Absorber
13a Absorbent core
13b Core wrap
13A Concave portion
13H High density section
13M Medium density section
13L Low density section
13V Density varying section
14 Top sheet
15 Side sheet
15G Raised gathering
15a Rubber thread
16 Fastening tape
16a Hook-and-loop fastener
17 Tape fastener sheet
18 Waist elastic sheet
18a Base sheet
18b Rubber thread
18c Upper sheet
19 Leg elastic member
19G Leg gathering
20a First recessed portion
20b Second recessed portion
20c Third recessed portion
201 Shallow recessed portion
202 Deep recessed portion
L1 A row of first recessed portions
L2 A row of second recessed portions
D1 First direction
D2 Second direction
S1 Interval between a row of second recessed portions
S2 Interval between a row of first recessed portions
Z_{R} Recessed portion forming area
Z_{N} Recessed portion non-forming area
Z_{S} Recessed portion semi-forming area
Z_{U} undeformed area
U Length of shallow recessed portion
V Width of shallow recessed portion
V1 Width of third recessed portion
Q1 Depth from top surface of top sheet in undeformed area to bottom surface of deep recessed portions
Q2 Depth from top surface of top sheet in undeformed area to bottom surface of shallow recessed portions
Q3 Height from bottom surface of shallow recessed portion to bottom surface of shallow recessed portion
Q4 Distance from top sheet to back sheet in undefined area (thickness of absorber)

1. An absorbent article having a liquid-permeable top sheet, a liquid-impermeable back sheet, and an absorber disposed between the top sheet and the back sheet, the absorbent article comprising:
a first recessed portion configured in a top surface of the absorber facing the top sheet and extending along a first direction; and
a second recessed portion configured in the top surface of the absorber facing the top sheet so as to intersect the first recessed portion, the second recessed portion extending along a second direction different from the first direction, and
wherein the first recessed portion and the second recessed portion each have a first area having a shallow depth from the top surface of the absorber to bottom surfaces of the first and second recessed portions, and a second area having a deeper depth from the top surface of the absorber to the bottom surfaces of the first and second recessed portions than the first area.

2. The absorbent article according to claim 1, further comprising a third recessed portion configured in an intersection area of the first recessed portion and the second recessed portion.

3. The absorbent article according to claim 2, wherein the third recessed portion is discontinuous with the first recessed portion and the second recessed portion.

4. The absorbent article according to claim 2, wherein the absorber has, in a peripheral portion thereof, a recessed portion non-forming area in which none of the first recessed portion, the second recessed portion, and the third recessed portion is configured.

5. The absorbent article according to claim 2, wherein the first recessed portion, the second recessed portion, and the third recessed portion are configured in the top sheet integrally with the absorber.

6. The absorbent article according to claim 3, wherein the first recessed portion, the second recessed portion, and the third recessed portion are configured in the top sheet integrally with the absorber.

7. The absorbent article according to claim 4, wherein the first recessed portion, the second recessed portion, and the third recessed portion are configured in the top sheet entegrally with the absorber.

8. The absorbent article according to claim 1, wherein a plurality of the first recessed portions is arranged at predetermined intervals along the second direction, and a plurality of the second recessed portions is arranged at predetermined intervals along the first direction.

9. The absorbent article according to claim 2, wherein a plurality of the first recessed portions is arranged at predetermined intervals along the second direction, and a plurality of the second recessed portions is arranged at predetermined intervals along the first direction.

10. The absorbent article according to claim 3, wherein a plurality of the first recessed portions is arranged at predetermined intervals along the second direction, and a plurality of the second recessed portions is arranged at predetermined intervals along the first direction.

11. The absorbent article according to claim 4, wherein a plurality of the first recessed portions is arranged at predetermined intervals along the second direction, and a plurality of the second recessed portions is arranged at predetermined intervals along the first direction.

12. The absorbent article according to claim 5, wherein a plurality of the first recessed portions is arranged at predetermined intervals along the second direction, and a plurality of the second recessed portions is arranged at predetermined intervals along the first direction.

13. The absorbent article according to claim 6, wherein a plurality of the first recessed portions is arranged at predetermined intervals along the second direction, and a plurality of the second recessed portions is arranged at predetermined intervals along the first direction.

14. The absorbent article according to claim 7, wherein a plurality of the first recessed portions is arranged at predetermined intervals along the second direction, and a plurality of the second recessed portions is arranged at predetermined intervals along the first direction.

15. The absorbent article according to claim 2, wherein the absorber has an undeformed area between the third recessed portion and each of the first recessed portion and the second recessed portion that are adjacent to the third recessed portion.

16. The absorbent article according to claim 3, wherein the absorber has an undeformed area between the third recessed portion and each of the first recessed portion and the second recessed portion that are adjacent to the third recessed portion.

17. The absorbent article according to claim 4, wherein the absorber has an undeformed area between the third recessed portion and each of the first recessed portion and the second recessed portion that are adjacent to the third recessed portion.

18. The absorbent article according to claim 5, wherein the absorber has an undeformed area between the third recessed portion and each of the first recessed portion and the second recessed portion that are adjacent to the third recessed portion.

19. The absorbent article according to claim 6, wherein the absorber has an undeformed area between the third recessed portion and each of the first recessed portion and the second recessed portion that are adjacent to the third recessed portion.

20. The absorbent article according to claim 7, wherein the absorber has an undeformed area between the third recessed portion and each of the first recessed portion and the second recessed portion that are adjacent to the third recessed portion.
